(19) United States
(12) Patent Application Publication
(10) Pub. No.: US 2011/0027315 A1
(43) Pub. Date: Feb. 3, 2011

(54) PROTEIN CAGES AND THEIR USES

(76) Inventors: Allen Harmsen, Bozeman, MT (US); Trevor Douglas, Bozeman, MT (US); Mark Young, Bozeman, MT (US)

Correspondence Address: COOLEY LLP;ATTN: Patent Group, Suite 1100, 777 - 6th Street, NW, WASHINGTON, DC, 20001, US

(21) Appl. No.: 12/676,797

(22) PCT Filed: Sep. 5, 2008
(86) PCT No.: PCT/US08/75373

§ 371 Date: Aug. 24, 2010

Related U.S. Application Data
(60) Provisional application No. 60/970,878, filed on Sep. 7, 2007.

(51) Int. Cl. A61K 39/145; A61K 39/00; A61K 39/12; A61K 38/02; A61P 31/04; A61P 37/04; A61P 11/06; A61P 31/16; A61P 31/12

(52) U.S. Cl. 424/210.1; 424/184.1; 424/204.1; 514/1.7

(57) ABSTRACT

The present disclosure provides compositions comprising a protein cage and methods for using the protein cage compositions to induce an immune protection response, or to prevent or ameliorate a viral or a bacterial infection.

### CROSS-REFERENCE TO RELATED APPLICATIONS

This application claims priority to U.S. Application Ser. No. 60/970,878 filed on Sep. 7, 2007, and to U.S. Application Ser. No. 60/986,985 filed on Nov. 9, 2007. The contents of U.S. Applications 60/970,878 and 60/986,985 are incorporated by reference herein in their entireties.

### BACKGROUND

There are several pathogens that cause severe and sometimes contagious diseases. Some of these pathogens are rapidly dividing pathogens, e.g., rapidly dividing lung pathogens. Examples of such pathogens include, but are not limited to those described below.

Bacillus anthracis: Anthrax is an acute infectious disease caused by the spore-forming bacterium Bacillus anthracis. Anthrax most commonly occurs in wild and domestic lower vertebrates (cattle, sheep, goats, camels, antelopes, and other herbivores), but it can also occur in humans when they are exposed to infected animals or to tissue from infected animals or when anthrax spores are used as a bioterrorist weapon.

Yersinia pestis: Pneumonic plague is a life-threatening infection of the lungs. It occurs when someone breathes in Yersinia pestis particles. Pneumonic plague is contagious, and an outbreak would be difficult to contain. It can be deadly if not treated quickly, preferably within 24 hours of the appearance of symptoms.

Francisella tularensis: Tularemia is a potentially serious illness that occurs naturally in the United States. It is caused by the bacterium Francisella tularensis found in animals (especially rodents, rabbits, and hares). If F. tularensis were used as a weapon, the bacteria would likely be made airborne for exposure by inhalation.

Brucella: Brucellosis is an infectious disease caused by the bacteria of the genus Brucella. These bacteria are primarily passed among animals, and they cause disease in many different vertebrates. Various Brucella species affect sheep, goats, cattle, deer, elk, pigs, dogs, and several other animals. Humans become infected by coming in contact with animals or animal products that are contaminated with these bacteria. In humans, brucellosis can cause a range of symptoms that are similar to the flu and may include fever, sweats, headaches, back pains, and physical weakness. Severe infections of the central nervous systems or lining of the heart may occur. Brucellosis can also cause long-lasting or chronic symptoms that include recurrent fevers, joint pain, and fatigue.

Streptococcus pneumoniae: Streptococcus pneumoniae (S. pneumoniae or “pneumococcus”) is a bacterium commonly found in the nasopharynx of healthy people. Occasionally, S. pneumoniae will spread from the nasopharynx of a colonized person into other parts of the body and cause diseases, including otitis media (ear infections), sinusitis (sinus infections) and pneumonia (lung infections).

Mycobacterium tuberculosis: Tuberculosis (TB) is a disease caused by bacteria called Mycobacterium tuberculosis. The bacteria usually attack the lungs. TB is spread through the air from one person to another. The bacteria are put into the air when a person with active TB disease of the lungs or throat coughs or sneezes. People nearby may breathe in these bacteria and become infected.

Pseudomonas aeruginosa: Pseudomonas aeruginosa is a bacterium responsible for severe nosocomial infections, life-threatening infections in immunocompromised persons, and chronic infections in cystic fibrosis patients. Pseudomonas aeruginosa, an increasingly prevalent opportunistic human pathogen, is the most common gram-negative bacterium found in nosocomial infections. P. aeruginosa is responsible for about 16% of nosocomial pneumonia cases.

Legionella pneumophila: Legionnaires' disease is caused by a type of bacteria called Legionella. The bacteria got its name in 1976, when many people who went to a Philadelphia convention of the American Legion suffered from an outbreak of this disease, a type of pneumonia (lung infection).

Nocardia asteroides: The bacterial complex Nocardia asteroides is a serious threat to immunosuppressed individuals, especially those with organ transplants, lung disease, and AIDS.

Staphylococcus aureus: Staphylococcus aureus causes chronic respiratory tract infections in patients with cystic fibrosis.

Mycoplasma pneumoniae: Mycoplasma pneumoniae is a small bacterium that can cause upper respiratory tract infections with fever, cough, malaise, and headache. Infections may lead to tracheobronchitis with fever and nonproductive cough: radiologically confirmed pneumonia develops in about 5-10% of cases.

Hemophilus influenzae: Hemophilus influenzae is one of several different bacteria that cause bacterial meningitis. Hemophilus influenzae Type B disease almost exclusively affects children under the age of five. The Hemophilus influenzae bacteria are spread from person to person by direct contact, or through sneezing or coughing.

Klebsiella pneumoniae: As a general rule, Klebsiella infections tend to occur in people with a weakened immune system. Many of these infections are obtained when a person is in the hospital for some other reason. The most common infection caused by Klebsiella bacteria outside the hospital is pneumonia.

Respiratory syncytial virus (RSV): Respiratory syncytial virus (RSV) is the most common cause of bronchiolitis and pneumonia among infants and children under 1 year of age. Illness begins most frequently with fever, runny nose, cough, and sometimes wheezing. During their first RSV infection, between about 25% and 40% of infants and young children have signs or symptoms of bronchiolitis or pneumonia, and about 0.5% to 2% require hospitalization.

Parainfluenza virus: Human parainfluenza viruses (HPIVs) are second to respiratory syncytial virus (RSV) as a common cause of lower respiratory tract disease in young children. Similar to RSV, HPIVs can cause repeated infections throughout life, usually manifested by an upper respiratory tract illness (e.g., a cold and/or sore throat). HPIVs can also cause serious lower respiratory tract disease with repeat infection (e.g., pneumonia, bronchitis, and bronchiolitis), especially among the elderly, and among patients with compromised immune systems.

Hantaviruses: Hantavirus pulmonary syndrome (HPS) is a disease from rodents. Humans can contract the disease when they come into contact with infected rodents or their urine and droppings. HPS was first recognized in 1993 and has since been identified throughout the United States. Although rare, HPS is potentially deadly. Rodent control in and around the home remains the primary strategy for preventing hantavirus infection.

SARS coronavirus: the SARS coronavirus is the virus that causes severe acute respiratory syndrome (SARS). On Apr. 16, 2003, following the outbreak of SARS in Asia and secondary cases elsewhere in the world, the World Health Organization (WHO) issued a press release stating that the coronavirus identified by a number of laboratories was the official cause of SARS.

Influenza virus: influenza is a common virus that causes considerable morbidity yearly and can cause severe morbidity and mortality upon periodic panepidemics. Efforts to develop effective vaccines to this virus have been hampered by the many seriologically distinct strains of the virus and its rapid mutation rate.

There is a need for methods of preventing the onset of diseases and/or infections caused by these and other pathogens.

Further, there is a need for reducing responses and symptoms that are part of chronic diseases, e.g., chronic pulmonary diseases, e.g., asthma or chronic obstructive pulmonary disease (COPD).

### SUMMARY

This disclosure is based, in part, on the discovery that protein cages, e.g., HSP protein cages, can be used as vaccines or vaccine components. The cages can also, or alternatively, prevent or ameliorate symptoms of infections or ameliorate symptoms of chronic diseases, such as asthma or COPD.

Accordingly, the present disclosure provides compositions comprising the protein cages and methods for using the protein cage to induce an immune protection response, to prevent or ameliorate a viral or a bacterial infection, and to prevent or ameliorate chronic diseases, e.g., chronic pulmonary diseases. Protein cage compositions can include one or more than one type of a protein cage. Methods utilizing protein cage compositions can likewise include administration of one or more than one type of a protein cage.

In one aspect, the disclosure features a composition, e.g., a vaccine composition, that includes a protein cage and an adjuvant.

Embodiments can include one or more of the following features.

The composition can include one or more than one, e.g., at least two, types of a protein cage (e.g., antigentic protein types, i.e., cages that can act as different antigens). The protein cage can be a self-assembled protein cage, and/or a viral protein cage. The protein cage can be a heat shock protein cage, a CCMV protein cage, a Dps protein cage, and a ferritin protein cage. The cage can contain one or more mutations, e.g., insertions, deletions, substitutions, or a combination thereof. For example, the protein cage can be a heat shock protein cage containing one or more mutations, e.g., G41C mutation. The adjuvant can include aluminum salts, endotoxins or other non-specific adjuvants, as well as antigens of pathogens, such as influenza virus-derived hemagglutinin and neuraminidase.

In another aspect, the disclosure features a method for inducing an immune protection response against a viral or bacterial infection. The method includes administering to a subject in need of such treatment an effective amount of a composition comprising a protein cage, wherein the subject develops an immune protection response against the viral or bacterial infection.

Embodiments can include one or more of the following features.

The composition can include one or more than one, e.g., at least two, types of a protein cage (e.g., antigentic protein types, i.e., cages that can act as different antigens). The protein cage can be a self-assembled protein cage, and/or a viral protein cage. The protein cage can be a heat shock protein cage, a CCMV protein cage, a Dps protein cage, and a ferritin protein cage. The cage can contain one or more mutations, e.g., insertions, deletions, substitutions, or a combination thereof. For example, the protein cage can be a heat shock protein cage containing one or more mutations, e.g., G41C mutation.

The composition can also include an adjuvant, e.g., aluminum salts, endotoxins, or other non-specific adjuvants, as well as antigens of pathogens, such as influenza virus-derived hemagglutinin and neuraminidase.

The immune protection response developed can include a localized lymphoid tissue in the subject, e.g., at a location where the composition is administered in the subject. The immune protection response can include induced localized collection of organized T cells, B cells, and plasma cells.

The administration can be directed to lung tissue, and the immune protection response can include developing a bronchus-associated lymphoid tissue. The administration can be topical or provided in an aerosol.

The viral or bacterial infection can be anthrax, tularemia, brucellosis, bacterial meningitis, otitis media, sinusitis, pneumonia, pneumonic plague, tuberculosis, bronchitis, bronchiolitis, wheezing, influenza, whooping cough, Legionnaires' disease, Hantavirus pulmonary syndrome, Severe Acute Respiratory Syndrome, and/or a nosocomial infection. The infection can be or can be associated with an infection of a lung and/or the pulmonary system. The viral or bacterial infection can be caused by at least one pathogen selected from the following group: influenza virus, respiratory syncytial virus (RSV), parainfluenza viruses, human parainfluenza viruses (HPIVs), hantaviruses, SARS coronavirus, Bacillus anthracis, Yersinia pestis, Francisella tularensis, Brucella, Streptococcus pneumoniae, Mycobacterium tuberculosis, Pseudomonas Aeruginosa, Legionella Pneumophila, Nocardia Asteroides, Staphylococcus aureus, Mycoplasma pneumoniae, Hemophilus influenzae, and Klebsiella pneumoniae.

The subject can be a mammal, e.g., a human.

In yet another aspect, this disclosure features a method of or for preventing or ameliorating a viral or a bacterial infection. The method includes administering to a subject in need of such treatment an effective amount of a composition comprising a protein cage, wherein the composition is administered in advance of the viral or the bacterial infection.

Embodiments can include one or more of the following features.

The composition can include one or more than one, e.g., at least two, types of a protein cage (e.g., antigentic protein types, i.e., cages that can act as different antigens). The protein cage can be a self-assembled protein cage, and/or a viral protein cage. The protein cage can be a heat shock protein cage, a CCMV protein cage, a Dps protein cage, and a ferritin protein cage. The cage can contain one or more mutations, e.g., insertions, deletions, substitutions, or a combination thereof. For example, the protein cage can be a heat shock protein cage containing one or more mutations, e.g., G41C mutation.

The composition can also include an adjuvant, e.g., aluminum salts, endotoxins, or other non-specific adjuvants, as well as antigens of pathogens, such as influenza virus-derived hemagglutinin and neuraminidase.

The composition can be administered at least three days, five days, seven days, or up to twenty one days before the viral infection or the bacterial infection. The composition can be administered in at least three doses over a two week period before the viral infection.

The viral or bacterial infection can be anthrax, tularemia, brucellosis, bacterial meningitis, otitis media, sinusitis, pneumonia, pneumonic plague, tuberculosis, bronchitis, bronchiolitis, wheezing, influenza, whooping cough, Legionnaires' disease, Hantavirus pulmonary syndrome, Severe Acute Respiratory Syndrome, and/or a nosocomial infection. The infection can be or can be associated with an infection of a lung and/or the pulmonary system. The viral or bacterial infection can be caused by at least one pathogen selected from the following group: influenza virus, respiratory syncytial virus (RSV), parainfluenza viruses, human parainfluenza viruses (HPIVs), hantaviruses, SARS coronavirus, Bacillus anthracis, Yersinia pestis, Francisella tularensis, Brucella, Streptococcus pneumoniae, Mycobacterium tuberculosis, Pseudomonas Aeruginosa, Legionella Pneumophila, Nocardia Asteroides, Staphylococcus aureus, Mycoplasma pneumoniae, Hemophilus influenzae, and Klebsiella pneumoniae.

The preventing or ameliorating can include preventing or ameliorating at least one symptom of the infection.

The subject can be a mammal, e.g., a human.

In another aspect, the disclosure features a method of ameliorating asthma in a subject in need thereof. The method includes administering to the subject an effective amount of a composition comprising a protein cage.

Embodiments can include one or more of the following features.

The composition can include one or more than one, e.g., at least two, types of a protein cage (e.g., antigentic protein types, i.e., cages that can act as different antigens). The protein cage can be a self-assembled protein cage, and/or a viral protein cage. The protein cage can be a heat shock protein cage, a CCMV protein cage, a Dps protein cage, and a ferritin protein cage. The cage can contain one or more mutations, e.g., insertions, deletions, substitutions, or a combination thereof. For example, the protein cage can be a heat shock protein cage containing one or more mutations, e.g., G41C mutation.

The ameliorating can include ameliorating at least one symptom of asthma, e.g., airway hyperreactivity. The subject can be a mammal, e.g., a human.

### BRIEF DESCRIPTION OF THE DRAWINGS

FIG. 1 is a graph showing the percent changes in initial body weight of Hsp-cage treated and vehicle treated mice following flu infection.

FIGS. 2A-2B are graphs showing the LDH and albumin levels in the broncho-alveolar lavage (BAL) fluids of the mice, before and after influenza infection.

FIG. 3 is a graph showing the amount of flu virus in the lungs of the four groups of mice at 7 days after infection, by determining flu plaque forming units (PFU).

FIGS. 4A-4C are graphs showing the levels of flu-specific IgA, IgG, and IgM in the BAL fluids of the mice, before and after influenza infection.

FIGS. 5A-5B are graphs showing the numbers of immune cells in the tracheal-broncheal lymph nodes (TBLN) and lungs (BAL) of the mice, 7 days after influenza infection.

FIG. 6A-D is a series of graphs, showing cell differentials at day 0 and day 7 after influenza infection.

FIG. 7 is a graph showing the total germinal center cells in the TBLN of infected mice.

FIG. 8A-8B are graphs showing the total CD4+ and CD8+ cells in both the BAL fluids and TBLN 7 days post flu infection.

FIGS. 9A-9D are graphs showing BAL cytokines IL-12p70, TNF, IFN-γ, and MCP-1, at day 0 and day 7 post flu infection.

FIGS. 10A-10B are graphs showing BAL cytokines IL-6 and IL-10, at day 0 and day 7 post flu infection.

FIG. 11 is graph showing the percent changes in initial body weight after flu infection in the Hsp-cage treated, and vehicle treated mice after 3 immunizations.

FIGS. 12A and 12B are graphs showing the total cell counts in the lungs (BAL) and TBLN after flu infection following 3 immunizations.

FIG. 13 is a graph showing the effect of 3 doses of low endotoxin-containing (1 ng/dose) Hsp cages on body weight changes after flu infection.

FIG. 14 is a graph showing the flu recovery after 3 doses of Hsp cages containing low levels of endotoxin.

FIG. 15 is a section showing iBALT in airway epithelium at 100× magnification following immunization but prior to flu infection.

FIG. 16 is a section showing nanoparticle-induced iBALT in C3H mice B cells and follicular dendritic cells.

FIG. 17 is an outline showing mice immunized intranasally twice a week for 4 weeks (total 9×).

FIG. 18 is an outline showing the immunization of mice intranasally twice a week for 4 weeks (total 9×) and data collected at day 0 and day 7.

FIG. 19 is a series of two sections showing BALT formation in the lung section immunized with HSPG41C protein cage.

FIG. 20 is a graph showing effects of heat shock protein nanoparticles on the survival of BALB/c mice exposed to an LD100 does of mouse-adapted SARS-CoV.

FIG. 21 is a graph showing effects of heat shock protein cage treatment on airway hyperreactivity of mice challenged intratracheally with ova.

FIG. 22 is a graph showing that full cage-induced protection lasts at least 21 days after treatment.

FIG. 23 is a graph showing that experiments carried out in different gene knockout mice indicate that adaptive immunity is required for protection.

FIG. 24 is a graph showing that in radiation chimeric mouse studies, cage-dependent protection is mediated through development of iBALT.

FIG. 25 is a graph showing that HSP cage treatment can protect mice from a low dose of an RSV-like paramyxovirus.

FIG. 26 is a graph showing that HSP cage treatment can protect mice from a high dose of RSV-like paramyxovirus.

### DETAILED DESCRIPTION

The present disclosure is based, in part, on the discovery that protein cages can be used to prevent or ameliorate infection, e.g., a bacterial and/or a viral infection. Thus, the cages can be used as a vaccine or a vaccine component. The cages can also be used to ameliorate symptoms of various inflammatory responses, e.g., airway inflammation, e.g., chronic or acute airway inflammation, e.g., inflammation and other symptoms associated with asthma or with COPD. The cages can be heat shock protein (HSP) cages, e.g., cages with specific mutations. The cages can be administered to a subject, e.g., a human subject, prior to an infection or prior to, during, or after an asthmatic reaction. When used as vaccines, the cages can be administered with an adjuvant. During use, one type of protein cage or more than one type can be administered to a subject. Administration can be carried out once or multiple times. When used as vaccines, the cages can help accelerate immune response to subsequent infection with a pathogen.

Protein Cages

Proteins can assemble, e.g., self-assemble, into three-dimensional and/or symmetric container-like, or cage structures, such as viral capsids or ferritin. Such cages have distinct interfaces that can be synthetically exploited: the interior, the exterior, and the interface between subunits. The subunits that comprise the building blocks of the cages can be modified, e.g., chemically and/or genetically, to impart designed functionality to different surfaces.

The protein cage described herein can be a self-assembled protein cage. In one embodiment, the protein cage is a viral protein cage. Examples of protein cages include, but are not limited to HSP protein cages, CCMV protein cages, Dps protein cages, and ferritin protein cages. The protein cages of the featured herein can contain one or more mutations, e.g., one or more insertions, deletions, substitutions, or a combination thereof.

In one embodiment, the protein cage is a heat shock protein (HSP or Hsp or hsp) cage, e.g., HSP cage containing one or more mutations. For example, the heat shock protein cage can contain a G41C mutation. The G41 residue is on the interior of the cage and allows for attachment of various molecules to the cage, e.g., fluorescent imaging agents, drugs etc. G41 residue can be chosen so that neighbouring residues in the cage are generally too far apart to form disulfide bonds. Other residues can be chosen for similar purposes.

HSPs are proteins whose expression is increased in response to elevated temperature or another type of stress, e.g., cold, oxygen deprivation, infection or inflammation. Some HSPs are present in the cells under normal conditions and have a chaperone function, e.g., they assist in proper folding of other proteins. They can be divided into several families, based on structure and function, e.g., HSP100, HSP90, HSP70, HSP60, and small HSPs. HSPs are somewhat resistant to denaturation because of some structural features, such as hydrogen bonds, hydrophobic internal packing, enhanced secondary structure, and helix dipole stabilization.

The small HSPs self-assemble from 24 identical subunits into a quaternary structure having cubic 432 symmetry with a hollow cage-like architecture. The subunits are identical and each is folded into a beta-sheet structure. The assembled cage has pores at both the 4-fold and the 3-fold axes, which can allow small molecules access to both the interior and the exterior of the structure. Various HSP families can be used with the methods and compositions described herein. In one embodiment, the small HSP family (16.5) from Methanococcus jannaschii can be used. For example, a Methanococcus jannaschii HSP cage structure includes 24 identical monomeric subunits of 147 amino acids, of 16.5 kDA, and of a 12-13 nm diameter sphere (Kim et al., Nature 1998, 394:595-599; Kim et al., J Struct. Biol. 1998, 121:76-80; Kim et al., PNAS 1998, 95:9129-9133.). In some embodiments, highly homologous proteins cloned from Sulfolobus solfataricus can be utilized and additional ones found in the sequence databases for many other archaea and bacteria.

Protein cages featured herein can be modified in various ways, e.g., specific residues can be mutated, and/or various moieties, e.g., antigens, opsinins, receptor agonists, and chemical moieties, attached via chemical or genetic means.

Protein cages featured herein can be generated by methods known in the art. For example, genetic constructs, e.g., DNA carrying plasmids, can be generated, expressed in desired hosts, e.g., bacteria and/or eukaryotic cells, and purified for administration to a subject, e.g., a human subject. Protein subunits can be modified chemically or genetically and can include mutations and/or chemical moieties.

Applications

Protein cages of this disclosure, e.g., HSP protein cages, can be used to induce immune responses and protect against infections, e.g., viral and/or bacterial infections, e.g., influenza, SARS, and RSV infections. Thus, the cages can be used as vaccines or vaccine components. The cages can also be used to ameliorate various symptoms of acute or chronic inflammation, e.g., to ameliorate airway hyperreactivity or other symptoms associated with asthma or to ameliorate symptoms of chronic obstructive pulmonary disease. One type of a cage or more than one type can be administered to a subject. Administration (of one or multiple cage types) can be carried out once or multiple times at various intervals.

Infections

This disclosure features methods of generating or inducing immune protection or immune response against a viral or a bacterial infection. The methods include administering to a subject, e.g., a human or an animal subject, in need of such treatment, an effective amount of a composition comprising a protein cage described herein, wherein the subject develops an immune protection or immune response against the viral or the bacterial infection. The composition, e.g., a vaccine, can, optionally, further include an adjuvant.

The disclosure also features methods of preventing or ameliorating symptoms of a viral or bacterial infection. The methods include administering to a subject, e.g., a human or an animal subject, in need of such treatment an effective amount of a composition that includes a protein cage described herein, wherein the composition is administered in advance of the viral or bacterial infection. The composition can also be administered after the viral or bacterial infection, if necessary. The subject can be evaluated for development or severity of the symptoms of an infection after the administration.

A vaccine is a composition formulated to improve immunity to a particular disease. It can be prophylactic or therapeutic. It often includes an adjuvant to help boost immune response.

The compositions of this disclosure, e.g., vaccine compositions, can be prepared for local or systemic administration. The compositions can be prepared for topical administration, e.g., as a solution, a gel, a suspension, a cream, or an ointment containing the protein cages described herein. The compositions can also be prepared for aerosol delivery, e.g., as a nasal spray, or they can be prepared for delivery from a pressurized aerosol canister. The compositions can be lyophilized, e.g., for long-term and/or large amount of storage. Storage in large amounts can be useful in case of an immediate need, such as during a pandemic (e.g., flu, RSV, and SARS pandemic). Lyophilized compositions can be administered as aerosols produced with a dust generator.

To immunize a subject, the protein cage or cages can be administered parenterally, usually by intramuscular or subcutaneous injection in an appropriate vehicle. Other modes of administration, however, such as oral delivery or intranasal delivery, are also contemplated by the present disclosure. Vaccine formulations will contain an effective amount of the active ingredient in a vehicle. The effective amount is an amount of composition that is sufficient to prevent, ameliorate or reduce the incidence of a particular infection (and/or its symptoms) in the subject (e.g., when compared to subjects, e.g., animal subjects, that did not receive the composition). The effective amount is readily determined by one skilled in the art and can be administered in one or more administration. The protein cage may typically range from about 1% to about 95% (w/w) of the composition, or higher or lower if appropriate. The quantity to be administered depends upon factors such as the age, weight and physical condition of the animal or the human subject considered for vaccination. The quantity also depends upon the capacity of the immune system to synthesize antibodies, and the degree of protection desired. Effective dosages can be readily established by one of ordinary skill in the art through routine trials establishing dose response curves. The subject is immunized by administration of the protein cage, e.g., HSP cage, in one or more doses. Multiple doses may be administered as is required to maintain a state of immunity.

The time period of administering the composition, the dosage, and the number of doses administered to the subject can vary. For example, the composition that includes protein cages is administered to the subject at least 1, 2, 3, 4, 5, 6, 7, 8, 9, 10, 12, 14, 16, 18, 21, 24 or 28 days before the viral or bacterial infection. Alternatively or in addition, the composition is administered in at least one, two, three, four or five doses over a two week period before the viral or bacterial infection. In one embodiment, the composition can be administered after the viral or bacterial infection to help ameliorate symptoms of the infection. The composition administered can include one type of a protein cage or more than one type of a protein cage. Thus, antigenically-distinct cages can be administered at the same time or at different times (e.g., if treatment with one cage is effective only once because of the immune response, an chemically-different cage can be used during another administration).

To prepare a composition, e.g., a vaccine composition, the purified protein cage, e.g., HSP cage, can be isolated, lyophilized and stabilized. The cage may then be adjusted to an appropriate concentration, optionally combined with a suitable vaccine adjuvant, and packaged for use. The adjuvant can be any adjuvant known to one of skill in the art, including, but not limited to, aluminum salts, endotoxins, and influenza virus-derived membrane-bound hemagglutinin and neuraminidase. For example, suitable adjuvants include but are not limited to surfactants, e.g., hexadecylamine, octadecylamine, lysolecithin, dimethyldioctadecylammonium bromide, N,N-dioctadecyl-N′-N-bis(2-hydroxyethyl-propane di-amine), methoxyhexadecyl-glycerol, and pluronic polyols; polanions, e.g., pyran, dextran sulfate, poly IC, polyacrylic acid, carbopol; peptides, e.g., muramyl dipeptide, MPL, aimethylglycine, tuftsin, oil emulsions, alum, and mixtures thereof. Other potential adjuvants include the B peptide subunits of E. coli heat labile toxin or of the cholera toxin. McGhee, J. R., et al., “On vaccine development,” Sem. Hematol., 30:3-15 (1993). The immunogenic product may also be incorporated into liposomes for use in a vaccine formulation, or may be conjugated to proteins such as keyhole limpet hemocyanin (KLH) or human serum albumin (HSA) or other polymers.

Intranasal formulations may include vehicles that neither cause irritation to the nasal mucosa nor significantly disturb ciliary function. Diluents such as water, aqueous saline or other known substances can be employed. The nasal formulations may also contain preservatives such as, but not limited to, chlorobutanol and benzalkonium chloride. A surfactant may be present to enhance absorption of the subject proteins by the nasal mucosa.

Oral liquid preparations may be in the form of, for example, aqueous or oily suspension, solutions, emulsions, syrups or elixirs, or may be presented dry in tablet form or a product for reconstitution with water or other suitable vehicle before use. Such liquid preparations may contain conventional additives such as suspending agents, emulsifying agents, non-aqueous vehicles (which may include edible oils), or preservative.

In one embodiment, the immune protection or immune response generated after protein cage administration includes developing a localized lymphoid tissue in the subject. For example, the immune protection response could include developing a lymphoid tissue at a location where the composition is administered in the subject. The immune protection response could also include inducing a localized collection of organized T cells, B cells, and plasma cells. Thus, the present methods provide for an immuno-prophylactic strategy that primes local immunity, e.g., lung or pulmonary immunity (innate and/or adaptive). It is thought that upon primary infection (in a subject naïve to the pathogen), the onset of the primary antibody response to the pathogen is accelerated, so that the virus-neutralizing antibodies can clear the virus before the virus proliferates to numbers sufficient to cause significant disease symptoms. This protection, mediated by the protein cages described herein, is effective against a wide range of viruses, e.g., lung or pulmonary viruses, and bacteria. In some embodiments discussed in the Examples below, the protection can last for at least three weeks after treatment.

For example, treatment with a protein cage, e.g., an HSP protein cage, can lead to development of bronchus-associated lymphoid tissue (BALT) in the lungs. Without being bound by theory, it is thought that this inducible BALT (iBALT) could augment and accelerate primary immune response to subsequently introduced pathogens, e.g., those that can cause pulmonary infections. Thus, in one embodiment, the administration of the compositions featured herein is directed to lung tissue, and the immune protection response includes developing a bronchus-associated lymphoid tissue.

The immune protection response induced or generated by the protein cages can be directed against any viral or bacterial infection. Similarly, vaccination or symptom amelioration can be sought against many viral or bacterial infections. Such infections include, without limitation, anthrax, tularemia, brucellosis, bacterial meningitis, otitis media, sinusitis, pneumonia, pneumonic plague, tuberculosis, bronchitis, bronchiolitis, wheezing, influenza, whooping cough, Legionnaires' disease, hantavirus pulmonary syndrome (HPS), Severe Acute Respiratory Syndrome (SARS), respiratory syncytial virus (RSV), or a nosocomial infection. The infection can be associated with a lung disease or a respiratory infection, for example, an influenza viral infection.

In general, the methods and compositions described herein induce an immune protection response against an infection by any pathogen, e.g., rapidly dividing pathogens. The methods also prevent, decrease, or ameliorate symptoms associated with infections by various pathogens. Examples of such pathogens include influenza viruses, respiratory syncytial virus (RSV), parainfluenza viruses, e.g., human parainfluenza viruses (HPIVs), hantaviruses, e.g., those that cause hantavirus pulmonary syndrome (HPS), SARS coronavirus, Bacillus anthracis, Yersinia pestis, Francisella tularensis, Brucella, Streptococcus pneumoniae, Mycobacterium tuberculosis, Pseudomonas aeruginosa, Legionella pneumophila, Nocardia asteroides, Staphylococcus aureus, Mycoplasma pneumoniae, Hemophilus influenzae, and Klebsiella pneumoniae.

The protein cages described herein can be effective against multiple types of pathogens, i.e., one type of a cage can provide protection against multiple strains of influenza, PMV or RSV, and coronavirus (e.g., SARS). Thus, treatment with the cages could be effective for first responders, regardless of genetic variations in a pathogen. The treatment could also be effective in causing maturation of infant respiratory immunity, e.g., to alleviate infant susceptibility to respiratory viruses, such as RSV. One of the advantages of the present methods is that pre-treatment/immunization with cages could augment a broad spectrum of immune mechanisms and minimize over-stimulation of a single mechanism that could cause adverse effects (for example, poly ICLC can induce an intense type I IFN production and cause flu-like symptoms).

Inflammation

The compositions that include protein cages, e.g., HSP protein cages, can also be used to prevent or ameliorate symptoms associated with chronic or acute inflammation, e.g., pulmonary inflammation. For example, the proteins cages can be administered to a subject who has asthma or COPD.

Asthma is a chronic condition of the respiratory system, in which the airways occasionally constrict, become inflamed, and fill with excessive amounts of mucus. Asthmatic attacks or episodes can be triggered by a variety of stimuli, e.g., allergens, tobacco smoke, exercise, or emotional stress. COPD is a lung disease that makes breathing difficult. It is caused by damage to the lungs, e.g., by long-term smoking. COPD is usually a mix of two diseases: chronic bronchitis and emphysema.

The compositions that include protein cages, e.g., HSP protein cages, can be administered before, during, or after a pulmonary episode, e.g., an asthma attack. The compositions can be administered to reduce such symptoms as airway hyperreactivity. The compositions can be administered in combination with other agents that ameliorate inflammation or other symptoms of asthma or COPD.

The compositions can be prepared for topical administration, e.g., as a solution, a gel, a suspension, a cream, or an ointment containing the protein cages described herein. The vaccine compositions can also be prepared for aerosol delivery, e.g., as a nasal spray, or they can be prepared for delivery from a pressurized aerosol canister. The compositions can be lyophilized, e.g., for long-term and/or large amount of storage. Lyophilized compositions can be administered as aerosols produced with a dust generator.

### EXAMPLES

The following examples are intended to illustrate, but not to limit, the disclosure. While they are typical of those that might be used, other procedures, methodologies, or techniques known to those skilled in the art may alternatively be used.

### Example 1

### Viral Cages Induce BALT

It was observed that certain viral cages behaved uniquely after being injected into mice. Small heat shock protein (sHsp) cages from Methanococus jannaschii were produced in an E. coli heterologous expression system, and Cowpea chlorotic mottle virus (CCMV) produced either from infected plant material or from a yeast (Pichia pastoris) heterologous expression system. These cages moved quickly through tissues, including readily crossing mucosal epithelium. In addition, because it was observed that multiple doses of the cages given to mice through the intravenous (i.v.) route caused adverse effects (anaphylaxis), determination of whether multiple doses of the cages injected directly into the lungs also caused adverse responses was carried out. Injecting cages into mouse lungs, twice a week for 4 weeks, did not cause adverse effects, unlike the i.v. injections. However, when histopathologic changes in the lungs of these mice were examined, it was found that the cage-treated mice had developed bronchus-associated lymphoid tissue (BALT). This inducible BALT (iBALT) consists of the same tissues and immune cells found in secondary lymphoid tissues such as the spleen and lymph nodes. In addition, it was found that this tissue could replace the function of secondary lymphoid tissues in mounting an immune response to pulmonary influenza infection. Thus, without being bound by theory, it is believed by the inventors that the presence of iBALT, as is induced with the cages, could augment the acquired immune response to pulmonary infections.

### Example 2

### Four Weeks of Treatment with Cages Protects Mice Against Influenza Infection

To determine whether cage treatment of mice could protect them from influenza infection, mice were treated twice a week for 4 weeks (29 days) with intratracheal injections of 100 μg/50 μL of HSP G41C cages or vehicle only (HBSS), for a total of 9 doses. The sHsp cages used were cloned from the Archaeon Methanococcus jannaschii and belonged to the ‘small hsp (16.5)’ family. Similar proteins from other Archaea, such as Sulfolobus solfataricus, which have high sequence homology and similar physical properties (i.e., they assemble into 24 subunit cages) have also been generated and will also be used.

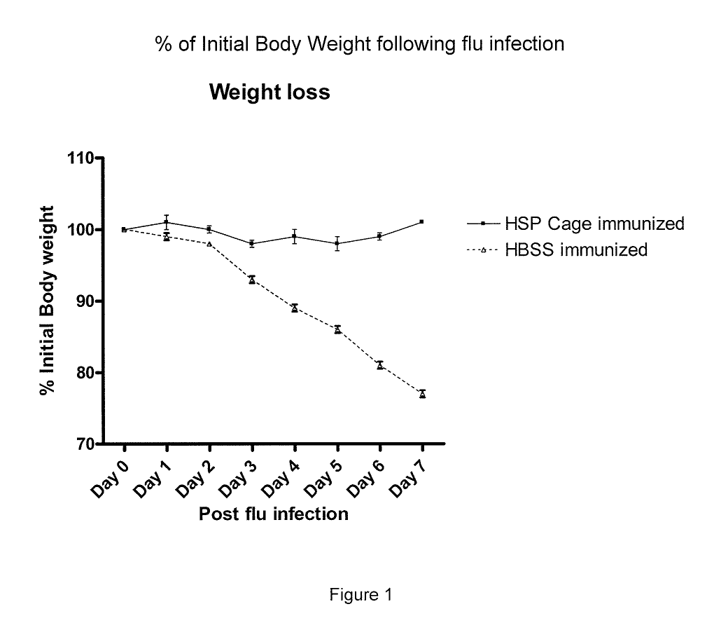

Forty eight hours after the last immunization, the mice were challenged with influenza strain PR8 at a dose just under the LD-50. The mice were weighed daily on the day of infection (time 0) until day 7 after flu infection and the results are shown on FIG. 1. The mice treated with the vehicle only (HBSS) gradually lost weight beginning on day one and had lost over 20% of their body weight by day 7 of infection. However, the mice pretreated with the Hsp cages did not lose any body weight after flu infection. The body weight changes of mice is one of the best indicators of the well being of animals after infection. Thus, the cage-treated mice appeared protected from the adverse effects of flu infection beginning already at day 1 after infection and extending for 7 days, suggesting that the cages augmented both the early innate immune response as well as the later acquired immune response.

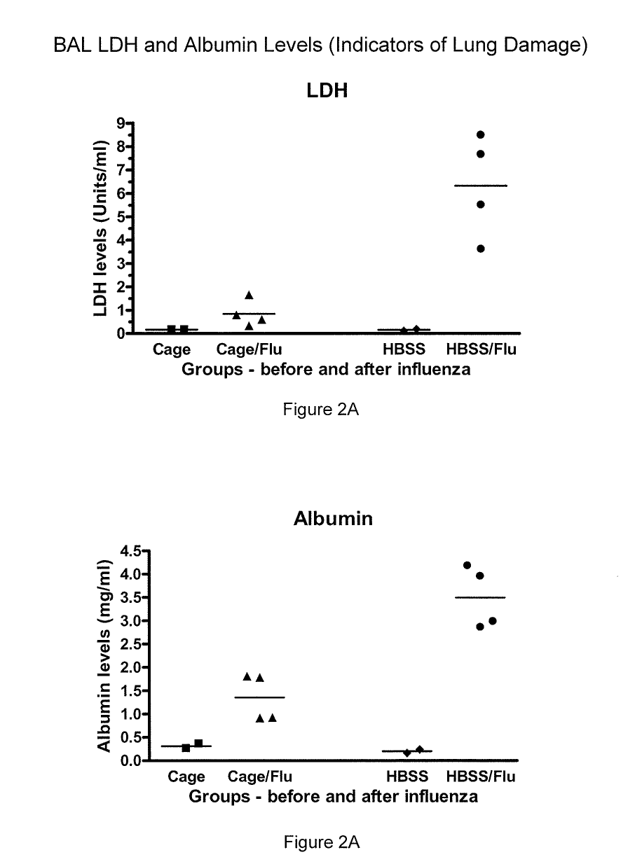

At day 7 after flu infection, the lungs of both groups of mice were also lavaged, and the broncho-alveolar lavage (BAL) fluids were assayed for LDH activity and for the presence of albumin. As shown in FIGS. 2A and 2B, the mice that received either cages only or vehicle (HBSS) only, had very low levels of LDH indicating that neither treatment caused damage in the lung since LDH is released from damaged cells. These two groups of mice also had low levels of albumin in the BAL fluids, indicating that no pulmonary edema was occurring in these mice. The mice treated with vehicle and infected with flu had high levels of both LDH and albumin, indicating lung damage in these mice as a result of the lytic flu virus infection. However, the mice treated with the cages before flu infection had LDH and albumin levels similar to those of the uninfected mice. Thus, indicators of both the well-being (body weights) and lung damage (LDH and albumin) were significantly improved in mice infected with flu if they were pretreated with the cages.

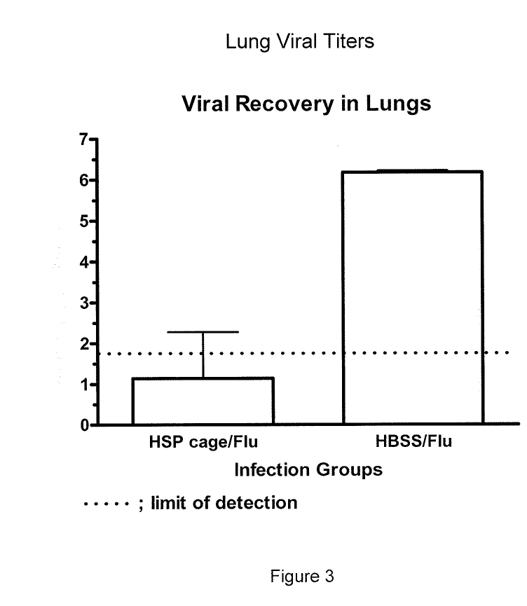

At 7 days after infection, the amount of flu virus in the lungs of the four groups of mice was analyzed, by determining flu plaque forming units (PFU) as shown in FIG. 3. Over one million flu PFU were found in the lungs of the flu-infected mice treated with vehicle, but less than 100 flu PFU were found in the lungs of the flu-infected mice pretreated with cages. Thus, HSP cage treatment reduced growth of virus in the lungs 100,000 fold. This indicates that the cage pretreatment resulted in augmentation of the clearance of flu particles from the lungs, possibly by augmentation of the flu-specific primary antibody response.

It was also found that mice are protected against other strains of influenza (H and N types). Animals treated with HSPG41C cage did not lose weight post-infection with X31 virus, and no virus could be detected in the lungs of HSPG41C-treated mice at day 7 post infection. The PBS control-treated mice lost 25% of their body weight by day 7 post-infection and had lung viral burdens of 2.5×10e5 pfus at day 7 post infection.

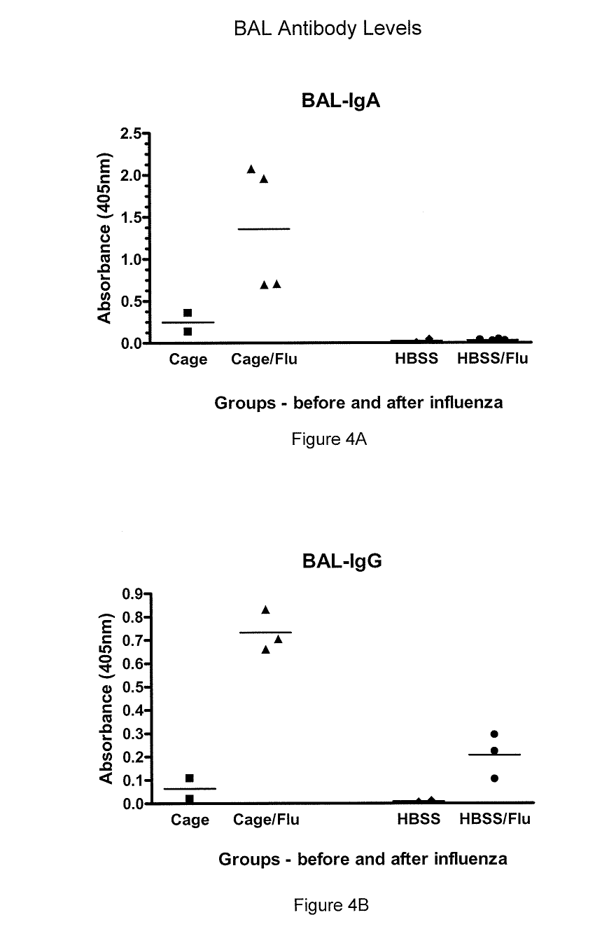

To determine whether the primary flu-specific antibody response was indeed augmented by the cage treatment, the levels of flu-specific IgA, IgG, and IgM in the BAL fluids were measured, and the results are shown in FIGS. 4A-4C. Both the flu-infected and the uninfected mice that received vehicle only had no detectable flu-specific IgA in their BAL fluids yet at day 7 of infection. However, the flu-infected mice pretreated with cages had significant titers of flu-specific IgA in their BAL fluids at 7 days (FIG. 4A). Similar results were seen with flu-specific IgG and IgM in BAL fluids (FIGS. 4B and 4C, respectively). It should be noted that the mice that received cages but not flu, had very low but detectable levels of flu-specific IgA and IgG. This could be due to higher background levels in these mice because the treatment with cages caused higher overall levels of antibodies, which would slightly increase the background signal in the ELISA assays.

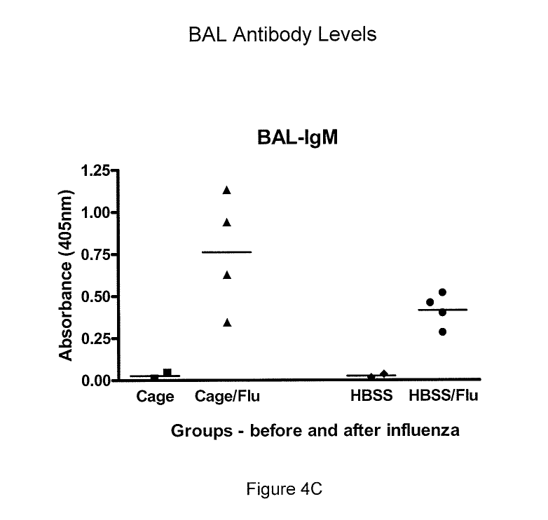

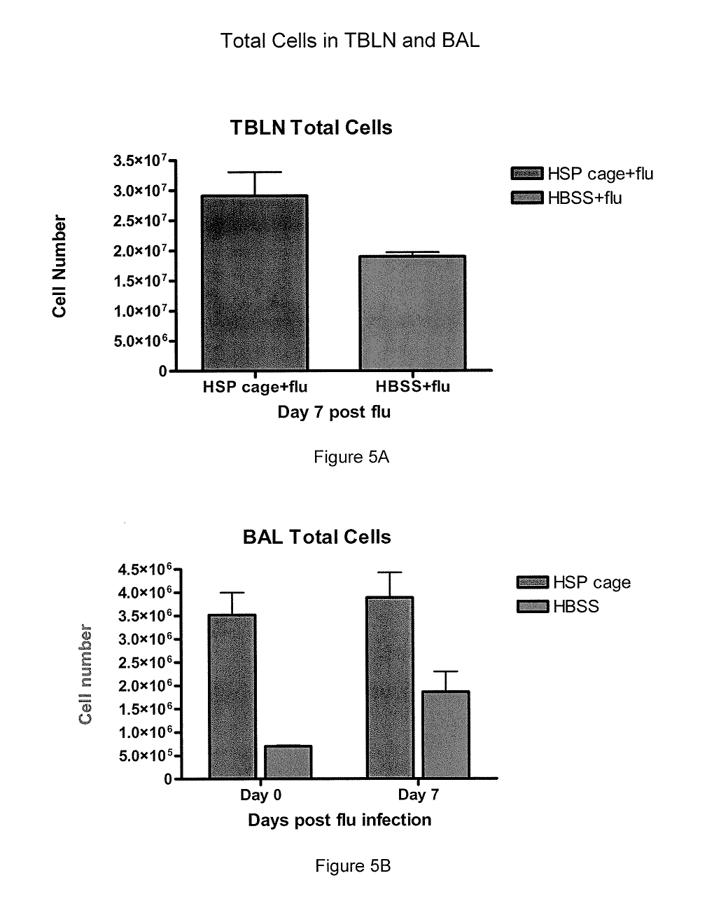

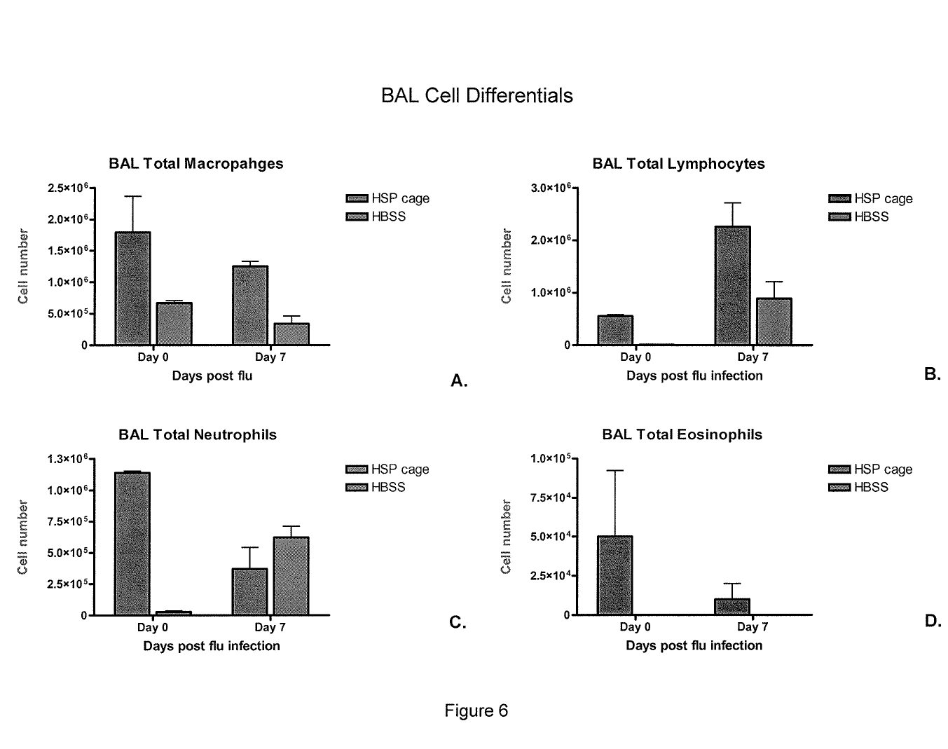

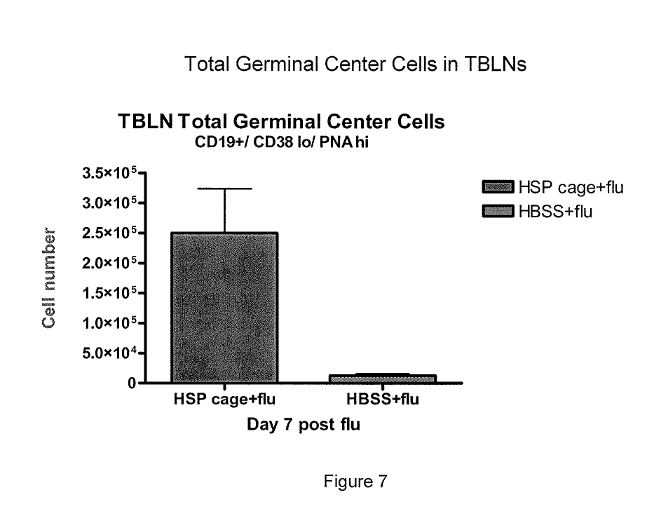

At day 7 of infection, the numbers of immune cells in the lungs and tracheal-broncheal lymph nodes (TBLN, which drain the lungs) of the mice were also determined. As seen in FIGS. 5A-5B, the cage treatment increased the immune cell content of both the TBLN and lungs. In the lungs, the increased cellularity at 7 days was mainly due to macrophage and lymphocyte accumulation (FIG. 6). Interestingly, the cage treatment resulted in a large increase in germinal center cells in the TBLN of infected mice (FIG. 7). This is consistent with the augmented antibody response in these mice. Also, the numbers of CD4 and CD8 T cells in both the BAL fluids and TBLN of the cage-treated mice infected with flu were greater than those numbers in flu-infected mice not treated with cages (FIGS. 8A-8B, respectively). Without being bound by theory, this suggests that besides augmentation of the antibody response, the cage treatment may also augment the cell-mediated response to the flu.

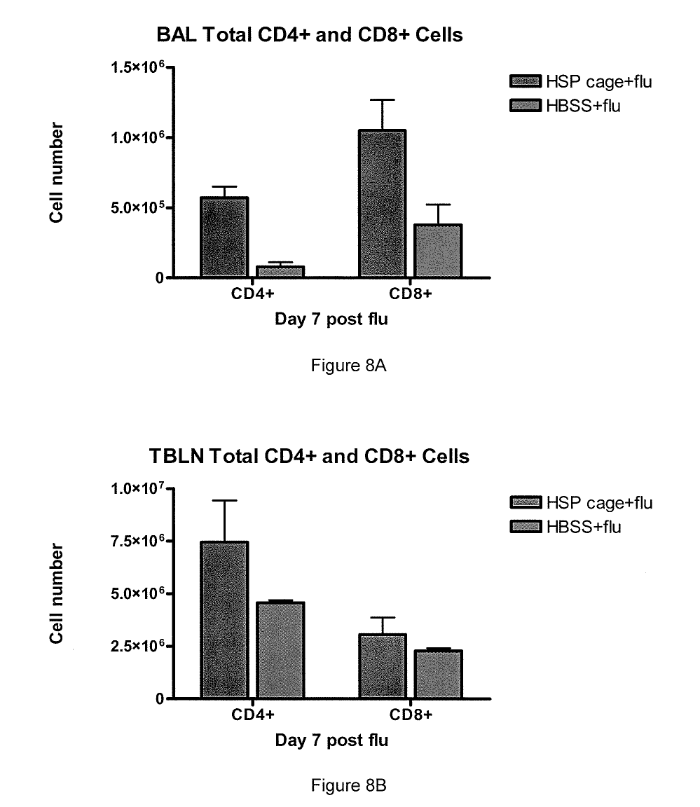

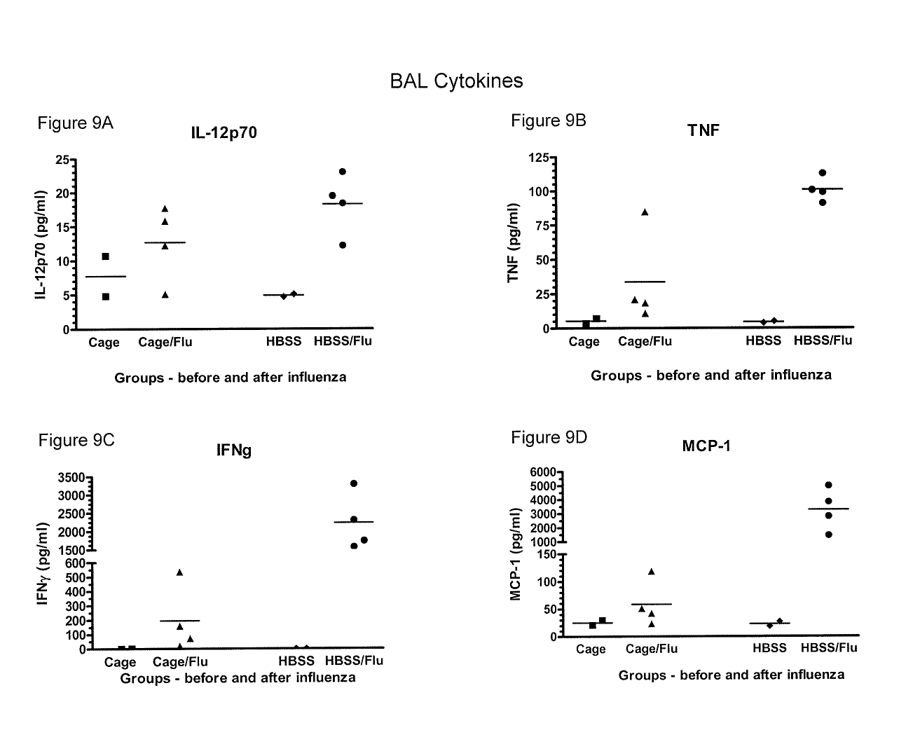

The levels of different cytokines in the BAL fluids were also determined in the 4 groups of mice at day seven of infection with flu. In general, the cage treatment reduced the levels of inflammatory cytokines in the lungs of the flu-infected mice, particularly TNF, IFN-γ, MCP-1, and IL-6 (FIGS. 9A-9D and 10B) as well as the anti-inflammatory cytokine IL-10 (FIG. 10A). This reduction in BAL cytokines was probably the result of the reduced viral burden in the cage-treated mice since the intensity of production of these cytokines are probably driven by the extent of virus burden.

### Example 3

### As Few as Three Doses of Cages Protect Mice Against Influenza Infection

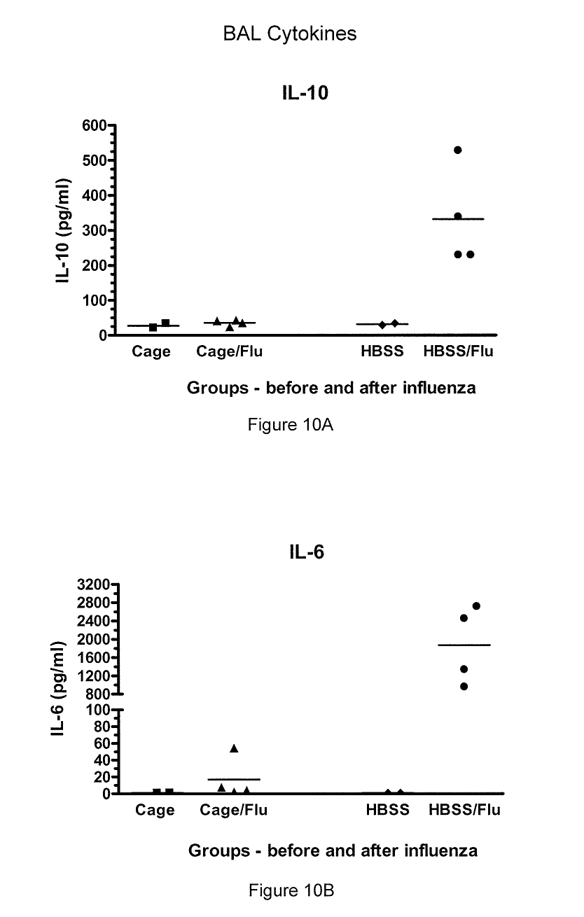

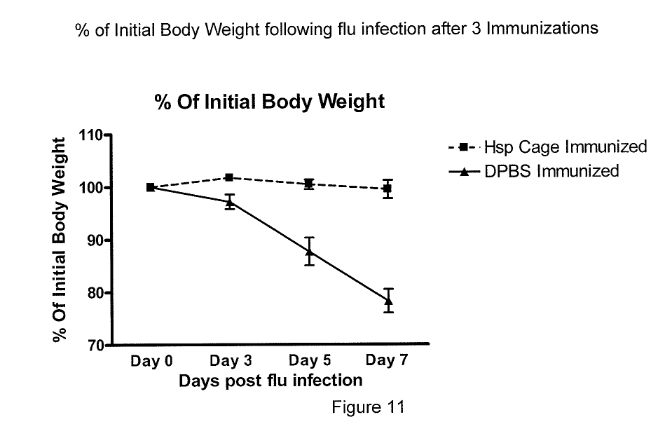

The initial experiments were done with 9 doses of cages, as the pretreatment. In order to determine whether fewer doses were also protective, mice were treated with intratracheal injections of either cages or vehicle only, twice in one week, then a third time the beginning of the next week, and then challenged the mice with flu at 3 days after the last treatment. The results of body weight changes in the two groups of mice after flu infection is shown in FIG. 11. Again, the mice treated with cages were protected from body weight loss caused by flu infection. The extent of protection was similar to that seen earlier after 9 doses with cages (as shown in FIG. 1). As in the first experiment with 9 doses, the 3 doses of cages reduced BAL LDH and albumin levels as well as flu virus titers (data not shown). In addition, similar results were seen for cage-induced increases in immune cell accumulation in the in the lungs and TBLN (FIGS. 12A and 12B, respectively).

To determine how long after treatment with 3 doses of cages the mice are protected, the animals were given 3 doses of either cages or vehicle and flu-challenged at 3, 10, 21, or 35 days after the last dose of cages or vehicle. We found that the mice challenged at either 10 or 21 days after treatment were protected to the same extent as those challenged at 3 days (data not shown). However, the animals challenged at 35 days after treatment had lost most of their cage-induced resistance to flu. Thus, the protection to flu induced by 3 doses of the cages persisted for about 21 days, after which time the protection diminished.

### Example 4

### Cage-Induced Protection Against Influenza Infection is Affected by Endotoxin within the Cage Preparations

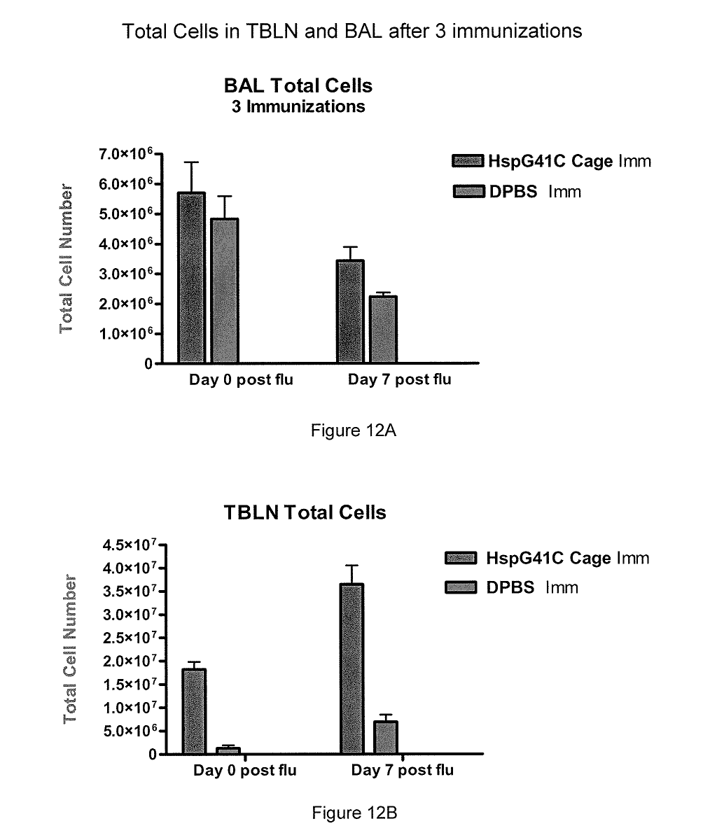

Because the Hsp cages are expressed in E. coli, the cage preparations could contain endotoxin. When endotoxin was quantified in the cage preparations, microgram amounts of endotoxin were found. Thus, some of the cage preparations were ran over a polymyxin B column to remove endotoxin, and the endotoxin level was lowered to 25.6 ng/ml. Because mice receive 50 μl of this preparation, this composition would be providing mice with 2.6 ng endotoxin, which is below the FDA-acceptable level for endotoxin contamination in therapeutics. The mice were dosed three times with this low endotoxin-containing cage preparation, or with vehicle, and both groups were challenged with flu. The cage-treated mice did lose some body weight after flu infection but significantly less than the vehicle treated mice (FIG. 13). This indicated that cages with such low endotoxin levels could induce partial protection to flu.

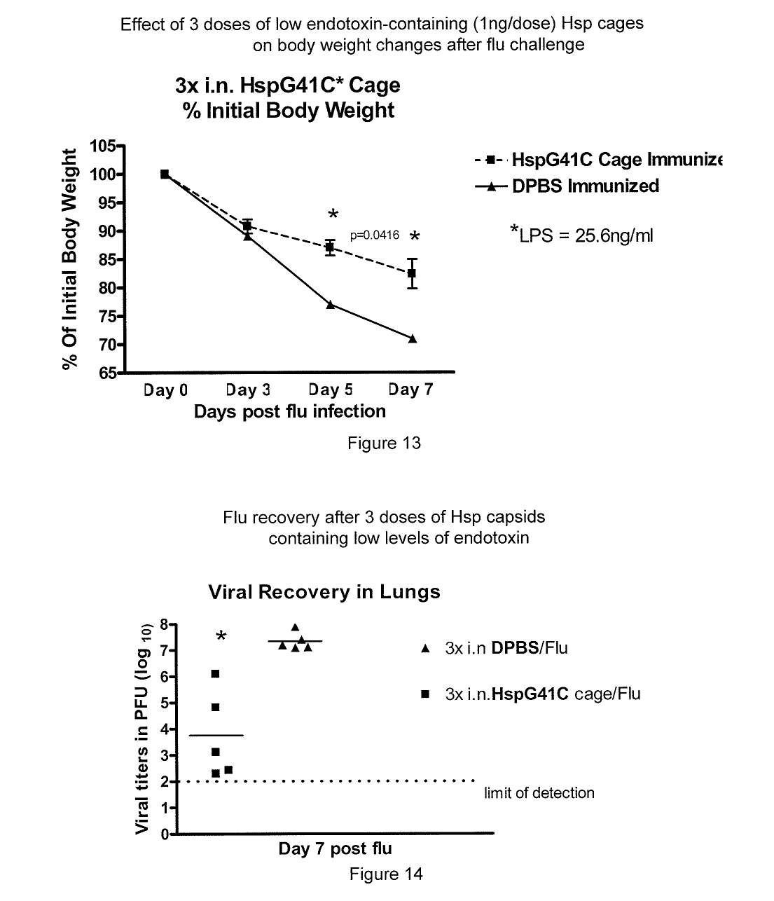

It was noticed that the cage-treated mice lost a similar amount body weight as the vehicle-treated mice during the first three days after infection. After three days, however, the cage-treated mice began to lose less weight. This is in contrast to the body weight lost curves seen in the previous experiments, where higher levels of endotoxin were present in the cage preparations. In those previous experiments, the cage-treated mice lost less weight than the vehicle-treated mice immediately after flu infection. This suggest that the endotoxin augmented innate defense mechanisms that resulted in early clearance (in the first 3 days) of virus, which did not occur in the mice given cages with low levels of endotoxin. Treatment with the low endotoxin-containing cages (1 ng/dose) did, however, significantly reduce flu virus titers (10-10,000-fold) as compared to the vehicle-treated mice (FIG. 14), but unlike the mice immunized with HSP containing endotoxin, viral recovery of influenza virus in the purified HSP immunized mice was above detection limits. Thus, the cages alone can augment the primary antibody and cell-mediated immune responses against influenza, and the inclusion of an adjuvant, such as endotoxin, probably augments innate defense mechanisms.

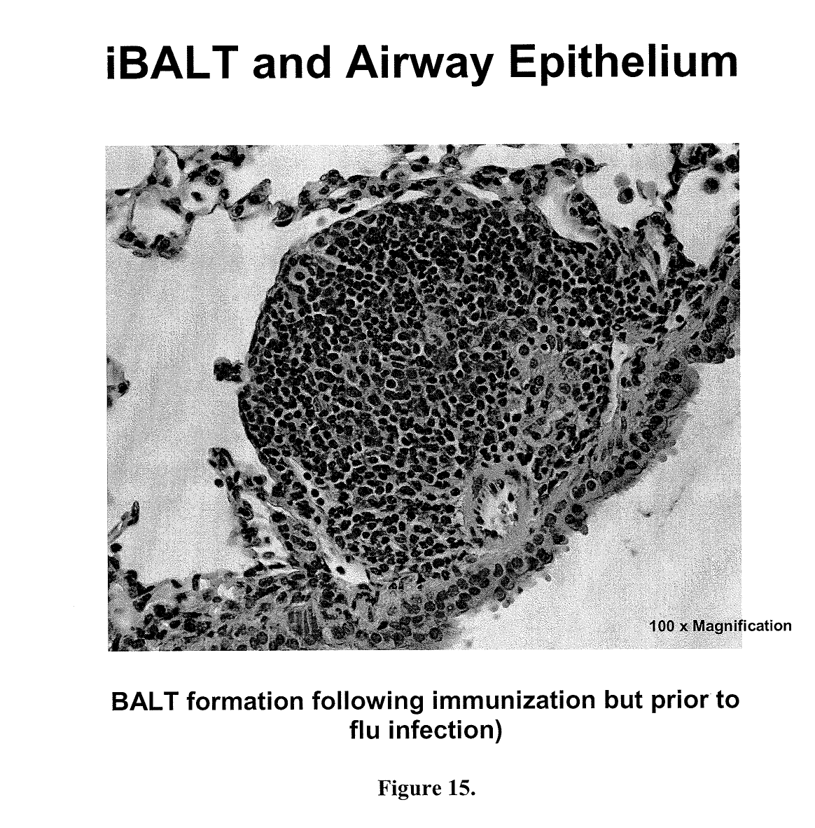

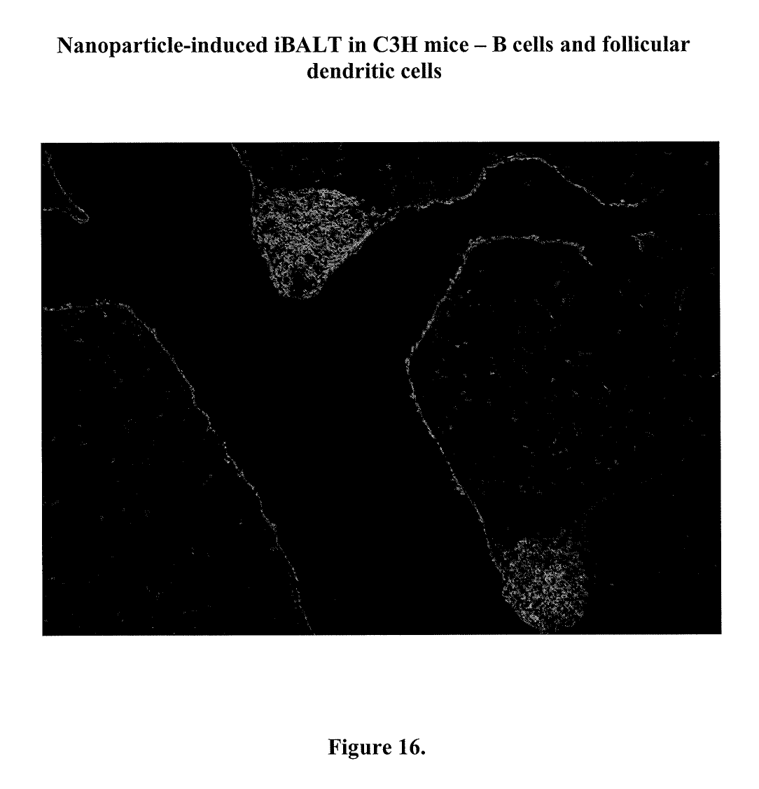

Without being bound by theory, it is thought by the inventors that augmentation of primary immune response to influenza by treatment with cages is due to a heightened local immune response. As discussed above, it was found that the cage treatment caused the development of iBALT (see also FIG. 15, showing iBALT and airway epithelium following immunization but prior to flu infection and FIG. 16, showing iBALT induced by 100 μg dose of HSPG41C nanoparticles (from Methanococcus jannaschii) in C3H mice: B cells show light staining at the edges and tips of the tissue and follicular dendritic cells show light staining at the tips of the tissue, intermixed with B cells). These tissues formed in peribronchiolar and perivascular locations in the lung, consisted of organized T and B cell areas, and included germinal centers, follicular dendritic cells, and adjacent tissue containing large numbers of plasma cells organized similarly to the medullary area of a lymph node. Without being bound by theory, it is thought that if iBALT preexists in the lungs, e.g., after being induced with cages, the primary immune response in a naïve host, occurs more quickly. The response may be quicker because it requires neither translocation of antigen to secondary distal lymphoid tissues nor elicitation of immune cells from those tissues to the lungs. Thus, the host with pre-formed iBALT can mount a protective primary response several days earlier than if iBALT was not pre-formed. This accelerated response can then clear a pathogen before it has had time to proliferate to sufficient numbers to cause significant damage in the lungs. The preexistence of iBALT is thought to favor the host immune response in the race between the proliferation of a potential pathogen and the development of a primary immune response against the potential pathogen. This represents a unique strategy of immunotherapy, which accelerates the host's primary immune response to a pathogen with antigens the host has not seen previously through either infection or immunization.

### Example 5

### Protein Cages with Mutated Subunits Provide Protection Against Influenza

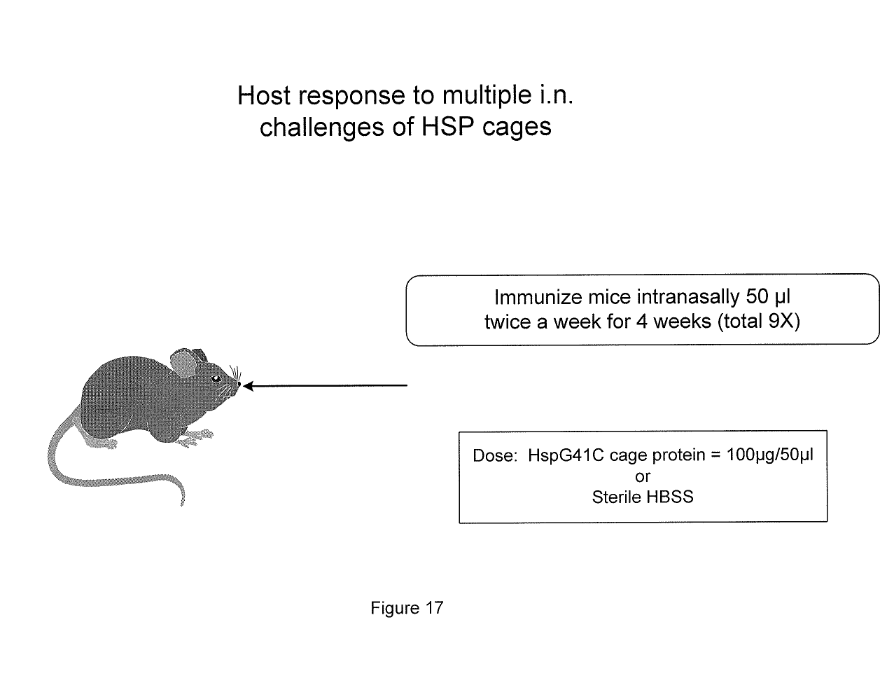

As shown in Example 1, mice immunized with a heat shock protein cage develop strong protection against influenza. In another experiment, mice were immunized intranasally twice a week for 4 weeks (9×) with 100 μg/50 μl of (a) heat shock protein cage (HSPG41C) or (b) sterile HBSS (FIG. 17). The HSP cage used was sHsp cloned from Methanococcus jannaschii into an expression vector and heterologously expressed in E. coli. The G41C mutation was generated by site-directed mutagenesis of the wild type gene. The G41C mutation was designed to provide a reactive group in the interior of the cage architecture without generally altering the exterior surface, and without generally disrupting the cage-like architecture.

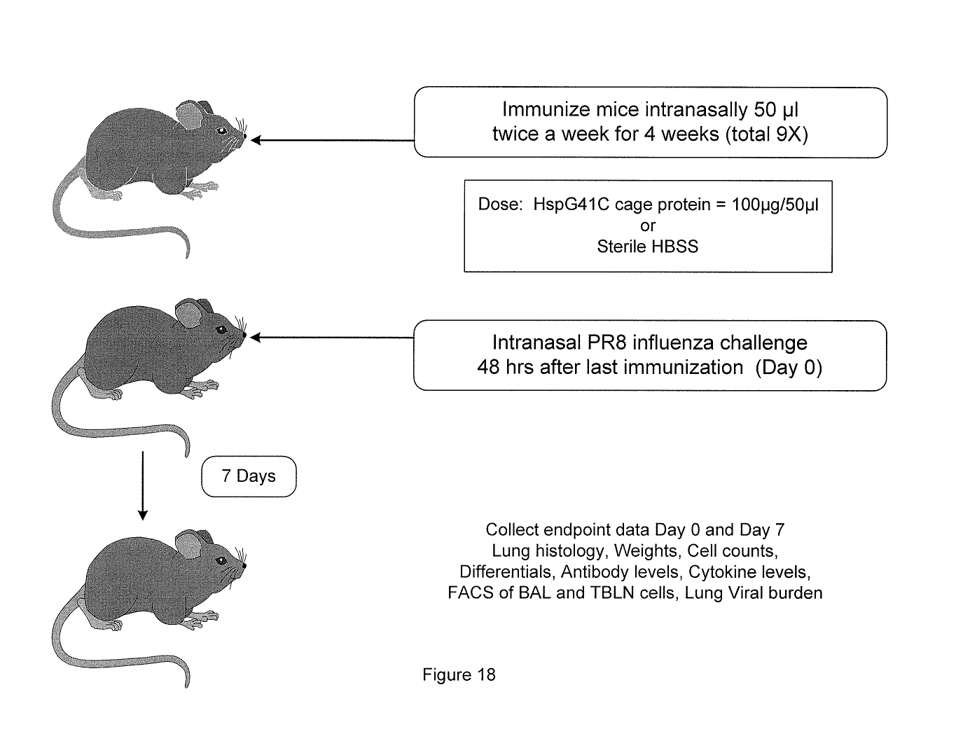

The mice were challenged with PR8 influenza 48 hours after the last immunization. Data was collected at Day 0 (time of challenge) and Day 7 (FIG. 18). The results, similar to those in Example 1, showed that the HSP inoculated mice had superior resistance to influenza as demonstrated by:

(a) No weight loss in the HSP immunized mice compared to HBSS immunized mice or HBSS or DPBS immunized mice;

(b) Significantly less lung damage in the HSP immunized mice compared to HBSS immunized mice;

(c) Below-detection recovery of influenza virus in the HSP immunized mice;

(d) Greater antibody levels in the HSP immunized mice compared to HBSS immunized mice;

(e) Greater total TBLN and BAL cells in the HSP immunized mice compared to HBSS immunized mice;

(f) Increased numbers of immune response related cells (macrophages, lymphocytes, neutrophils, eosinophils, germinal center cells, CD4+ and CD8+ T cells) in HSP immunized mice compared to HBSS immunized mice; and

(g) Greater manifestation of influenza in the HBSS immunized mice compared to the HSP immunized mice as indicated by greater numbers of cytokines in the HBSS immunized mice following flue infection.

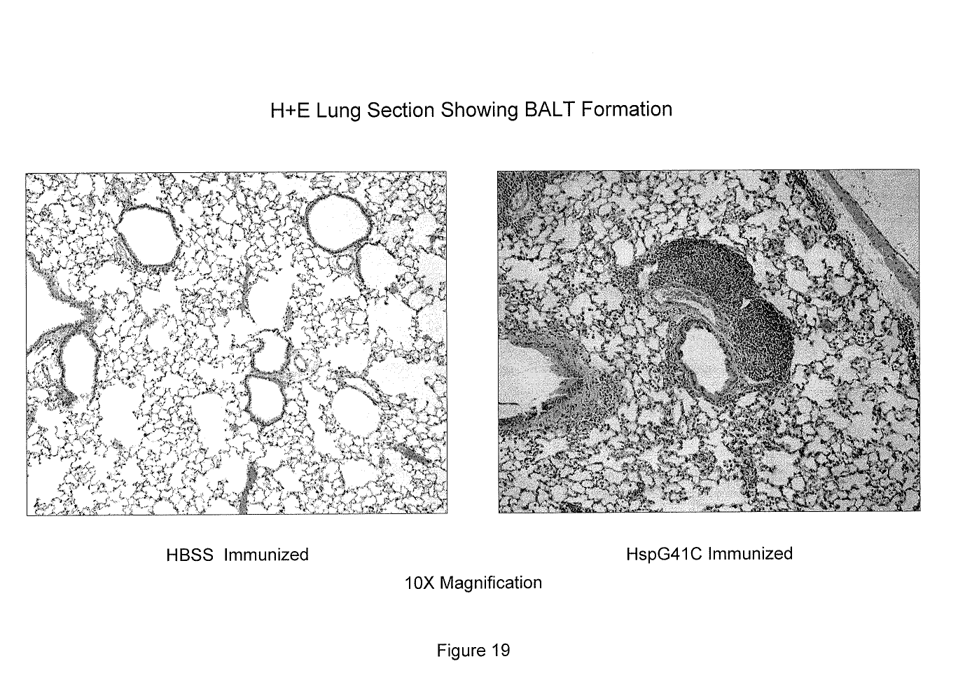

Further, BALT cells (which normally appear in the lung within 5 days of exposure to influenza) were shown in HSP mice to appear in 3 days, demonstrating accelerated localized immunity (lymph node-like structures) in the lung (FIG. 19).

### Example 6

### HSP Cages Protect Mice Against SARS Infection

Three groups of mice were challenged with 1LD100 (dose lethal to 100% of animals) of mouse-adapted SARS-CoV virus intratracheally. One of the three groups was additionally treated with vehicle only (PSS: phosphate saline solution, negative control), a second group was additionally treated with poly ICLC (Polyinosinic-Polycytidylic acid stabilized with polylysine and carboxymethylcellulose, positive control at 5 mg/kg/day), and a third group was additionally treated with HSPG41C nanoparticle (5 doses over two weeks of 5 mg/kg/day or 100 μg/dose). Poly ICLC can be used as anti-SARS therapy in humans, but can cause flu-like symptoms. The HSPG41C particles were the Methanococcus jannaschii HSPs, incorporating the G41C mutation, heterologusly expressed in E. coli, and purified to homogeneity.

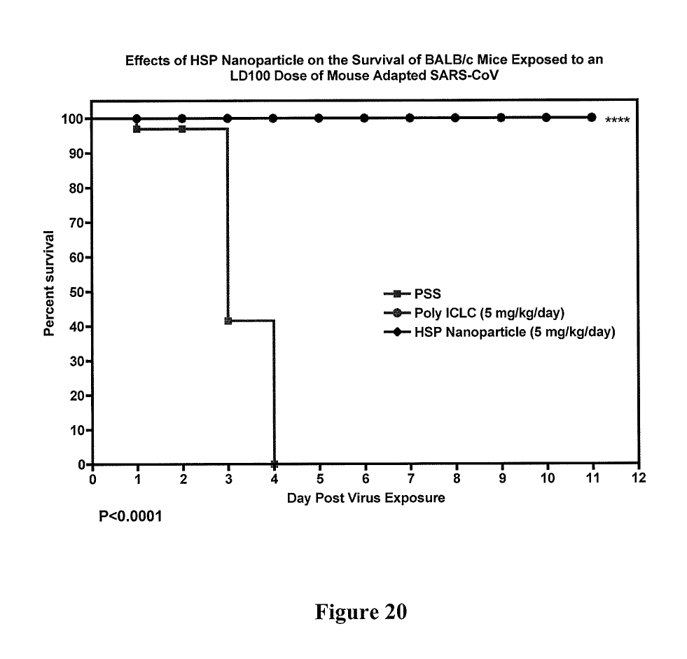

As shown in FIG. 20, mice that were given vehicle only all died by day 4 after viral exposure. Mice that were given either HSP cages or Poly ICLC all survived (the two traces are superimposed). The mice treated with Poly ICLC lost a considerable amount of weight when compared with the HSP-treated mice (data not shown).

### Example 7

### HSP Cages Ameliorate Asthma

Three groups of mice were challenged with methacholine (which can be used to diagnose asthma). One group was not sensitized with ovalbumin (ova) and received vehicle only; one group was ova sensitized and received PBS (phosphate buffered saline, a positive control); and the last group was ova sensitized and received HSPG41C cages (5 doses of 100 μg over two weeks). The HSPG41C particles were the Methanococcus jannaschii HSPs, incorporating the G41C mutation, heterologusly expressed in E. coli, and purified to homogeneity.

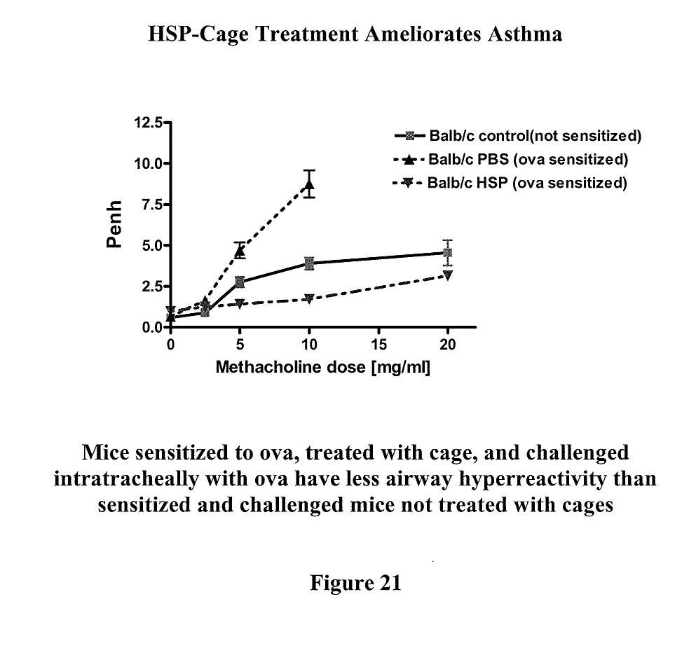

As shown in FIG. 21, ova sensitized group that received PBS had a high level of airway hyperreactivity upon methacholine challenge, the group that was not sensitized and did not receive any treatment had an intermediate level of airway hyperreactivity, and the HSP treated and ova sensitized group of mice had a low level of airway hyperreactivity. The Y axis shows Pₑₙₕ: “enhanced pause,” which is an assessment of airway resistance that can increase with lung hypersensitivity.

### Example 8

### Cage-Induced Protection Lasts at Least 21 Days after Treatment

Mice were challenged with HSPG41C cage (100 μg per dose) or vehicle on days 3, 10, 21, or 35 prior to flu infection. The HSPG41C particles were the Methanococcus jannaschii HSPs, incorporating the G41C mutation, heterologusly expressed in E. coli, and purified to homogeneity.

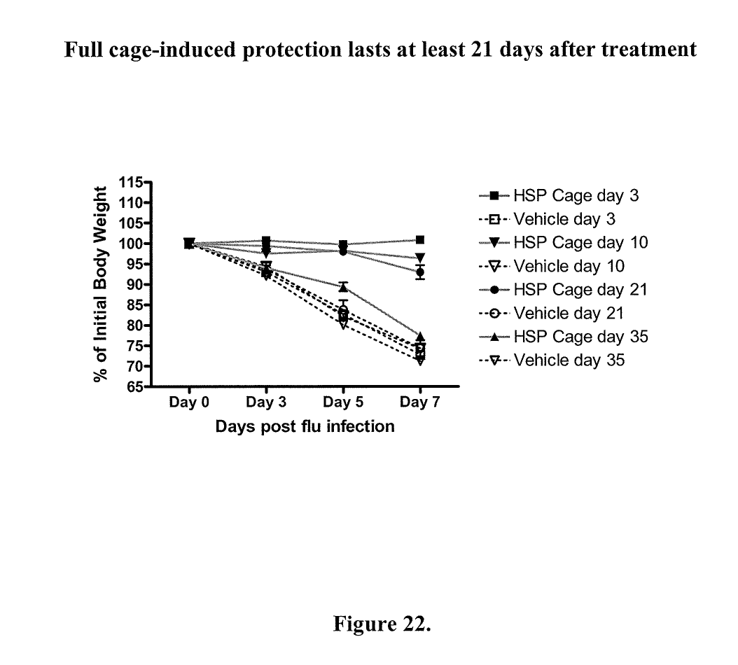

As shown in FIG. 22, after flu infection (with PR8 influenza [H1N1]), mice treated with HSP at days 3, 10, and 21 showed a smaller (if any) decrease in percent of body weight, when compared with vehicle-treated groups or with the group treated at day 35.

### Example 9

### Protection can be Mediated Via Adaptive Immunity

To analyze the involvement of adaptive immunity in the cage protection, various knockout mice were pre-treated with HSPG41C cages (at 100 μg/dose for 5 doses over 2 weeks) or PBS vehicle before infection with flu. The HSPG41C particles were the Methanococcus jannaschii HSPs, incorporating the G41C mutation, heterologusly expressed in E. coli, and purified to homogeneity.

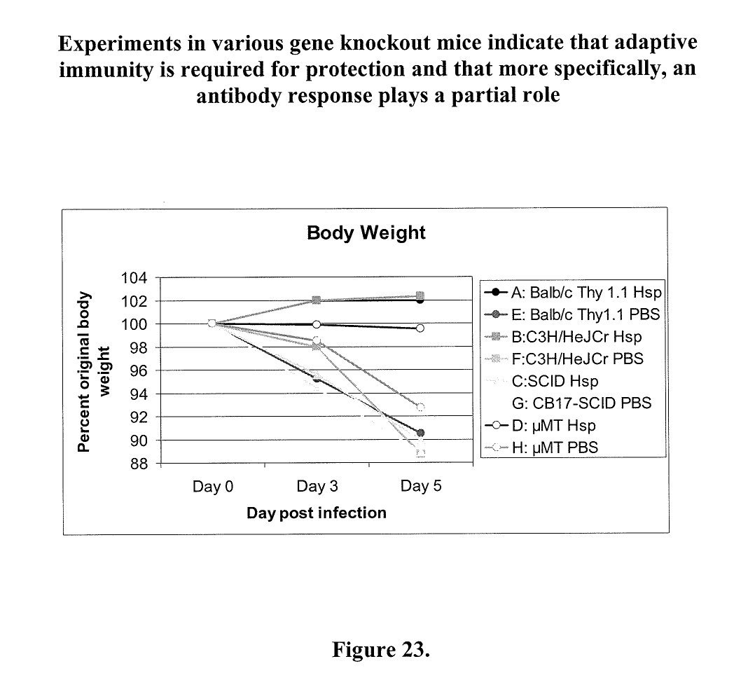

As shown in FIG. 23, mice that did not show a large decrease in body weight after infection included the following knockouts treated with HSP: Balb/c Thy 1.1 (Thy 1.1 is a marker of mouse mature T lymphocytes), C3H/HeJ Charles River (these mice carry a mutation in toll-like receptor 4 gene, Tlr4 lps, making them endotoxin resistant; therefore the response of these mice is due to the HSP rather than endotoxin), and μMT (these mice lack functional T and B lymphocytes). The PBS-treated controls of these three knockout groups lost weight. Both the control and the HSP-treated SCID mice lost much weight, showing no protection by HSP cages. These results show that adaptive immunity is involved in HSP cage protection. More specifically, an antibody response plays a partial role, as the μMT knockouts were partially protected.

### Example 10

### Cage-Dependent Protection is Mediated Through Development of iBALT

To further analyze the involvement of iBALT in the cage-dependent protection, Ltα−/− mice were used in bone marrow transplantation experiments. Four groups of mice were generated: (1) wild type mice were irradiated, received transplantation of wild type bone marrow cells, were pre-treated with PBS, and then were infected with flu; (2) wild type mice were irradiated, received transplantation of wild type bone marrow cells, were pre-treated with HSPG41C cages, and then were infected with flu; (3) Ltα−/− mice (which have a T cell deficiency) were irradiated, received transplantation of wild type bone marrow cells, were pre-treated with PBS, and then were infected with flu; and (4) Ltα−/− mice were irradiated, received transplantation of wild type bone marrow cells, were pre-treated with HSPG41C cages, and then were infected with flu. Mice pre-treated with HSP received HSPG41C at 100 μg/dose for 5 doses over two weeks. For irradiation, a cesium source whole body irradiator was used for 900 Gys of gamma irradiation (administered in a split dose of 475 Gy each, 4 hours apart). The HSPG41C particles were the Methanococcus jannaschii HSPs, incorporating the G41C mutation, heterologusly expressed in E. coli, and purified to homogeneity.

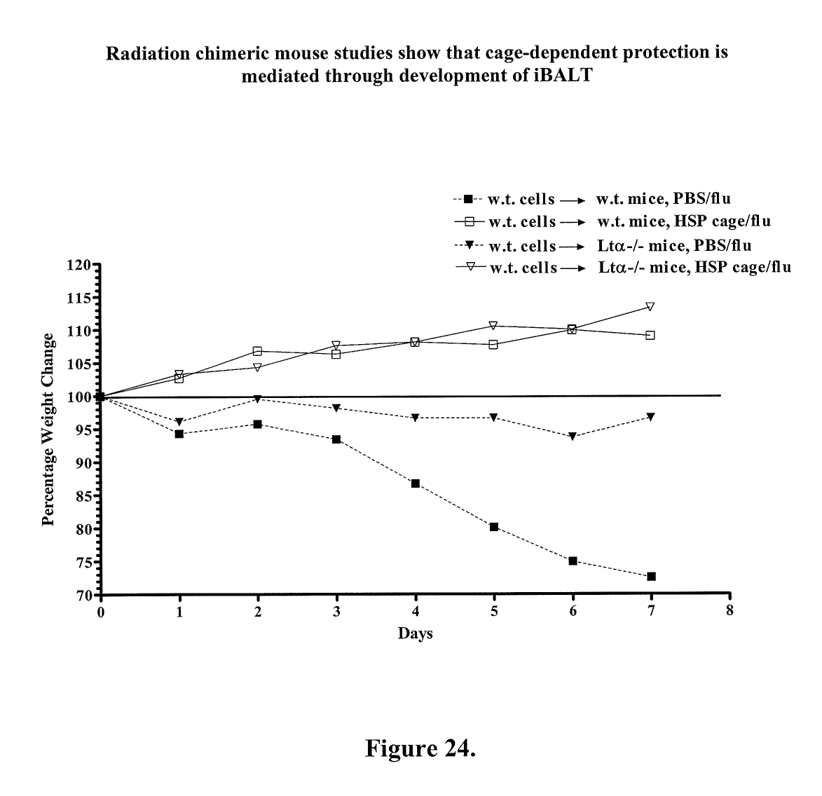

As shown in FIG. 24, both the wild type mice treated with HSP cages and the Ltα−/− mice who were treated with HSP and received a wild type population of T cells did not show a decrease in body weight, following flu infection.

### Example 11

### HSP Cage Treatment Protects Mice from RSV-Like Paramyxovirus

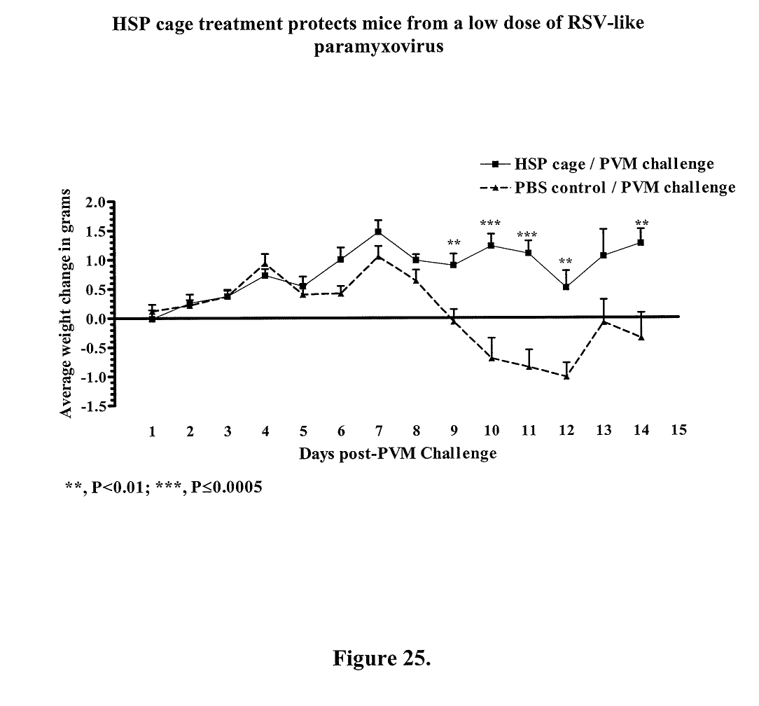

To analyze HSP protection against a paramyxovirus, which is an RSV-like virus, mice were pre-treated/immunized with either a PBS control or HSPG41C cage (administered in 4 or 5 doses, intranasally in a 50 μL volume, at 100 μg per dose). Both groups were challenged with paramyxovirus (PVM, administered intranasally 160×LD50 in a 80 μL volume) 4 days after the last HSP dose and their weight changes analyzed. As shown in FIG. 25, at day 9, post-infection, control mice began losing significantly more weight than their HSP-treated counterparts. Thus, this data shows that HSP cage treatment can protect mice from a low dose of an RSV-like paramyxovirus.

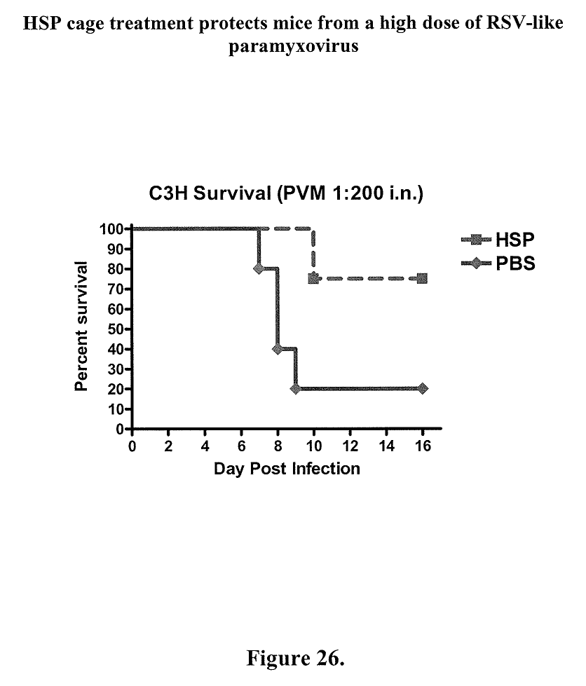

To analyze HSP protection against a high does of PVM, C3H mice were pre-treated with either HSP (at 100 μg/dose for 5 doses over two weeks, administered intranasally in a 50 μL volume) or PBS and infected with PVM (1:200 dilution of the stock virus to obtain 160×LD50). As shown in FIG. 26, mice treated with HSP had a higher survival rate than the control group.

### Example 12

### Cage Treatment does not Show Adverse Effects

Various homeostatic serum metabolism parameters of mice treated with cages were analyzed for any adverse effects of cage treatment. Tables 1 and 2 below show an array of nephritic and hepatic enzyme panels of groups of PCN-treated (HSP-treated) mice at 12 and 48 hours following a 100 μg dose of PCN. Renal and hepatic blood chemistry profiles were normal at both 12 hours and 48 hours after the cages were injected intravenously. The cages were also nearly completely cleared from the host by 12 hours, and the treatment did not increase (it decreased) susceptibility to lung hypersensitivity (data not shown). Serum levels of sodium, calcium, chloride, potassium and phosphorous were measured and found to be unaffected by the PCN treatment. No statistically significant differences were seen for alanine transferase, glucose, cholesterol, blood urea nitrogen, total protein, albumin, serum Ig and creatine levels between the PCN and PBS treated groups. Lower levels of alkaline phosphatase were detected in the serum of the PCN-treated mice at 12 and 48 hours after treatment. In chronic clinical diseases, such as scurvy and malnutrition, decreased serum levels of alkaline phosphatase are generally expected. However, since the parameters measured herein were acute responses and chronic diseases associated with decreased alkaline phosphatase levels were not present in the studied mice, it is unlikely that lower levels of alkaline phosphatase are indicative of an adverse toxicological reaction to the PCN treatment.

TABLE 1
Metabolic Parameters Measured 12 hours After HSP Cage Treatment

| | | | | | | Blood | | | | |
|---|---|---|---|---|---|---|---|---|---|---|
| | Asapartate | Alanine | Alk. | | | Urea | Total | | | |
| | Aminotrans | Aminotrans | Phos. | Glucose | Cholesterol | Nitrogen | Protein | Albumin | Ig | Creatine |
| | (IU/L) | (IU/L) | (IU/L) | (mg/dl) | (mg/dl) | (mg/dl) | (g/dl) | (g/dl) | (g/dl) | (mg/dl) |
| PBS | 86.6 ± 27.6 | 37.3 ± 12.5 | 149.8 ± 10.0 | 110.6 ± 15.9 | 107.6 ± 6.2 | 28.3 ± 3.6 | 5.6 ± 0.1 | 3.5 ± 0.2 | 2.2 ± 0.2 | 0.2 ± 0.05 |
| contr. | | | | | | | | | | |
| HSP | 125.6 ± 64.5* | 52.6 ± 63.6 | 100.4 ± 8.9** | 59.8 ± 10.2** | 120.7 ± 9.0 | 30.2 ± 2.7 | 5.8 ± 0.2 | 3.5 ± 0.2 | 2.3 ± 0.2 | 0.2 ± 0.07 |
| cage | | | | | | | | | | |

TABLE 2
Metabolic Parameters Measured 48 hours After HSP Cage Treatment.

| | | | | | | Blood | | | | |
|---|---|---|---|---|---|---|---|---|---|---|
| | Asapartate | Alanine | Alk. | | | Urea | Total | | | |
| | Aminotrans | Aminotrans | Phos. | Glucose | Cholesterol | Nitrogen | Protein | Albumin | Ig | Creatine |
| | (IU/L) | (IU/L) | (IU/L) | (mg/dl) | (mg/dl) | (mg/dl) | (g/dl) | (g/dl) | (g/dl) | (mg/dl) |
| PBS | 81.8 ± 16.7 | 47.8 ± 13.6 | 187.0 ± 1.0 | 143.0 ± 23.3 | 104.9 ± 17.3 | 45.1 ± 33.0 | 5.5 ± 0.2 | 3.7 ± 0.3 | 1.9 ± 0.2 | 0.6 ± 0.06 |
| contr | | | | | | | | | | |
| HSP | 73.4 ± 9.7 | 33.6 ± 4.6 | 122.3 ± 3.6** | 126.3 ± 7.8 | 120.9 ± 17.4 | 30.5 ± 4.0 | 5.4 ± 0.2 | 3.2 ± 0.1 | 2.2 ± 0.1 | 0.3 ± 0.06 |
| cage | | | | | | | | | | |

Thus, adverse effects were generally not seen in the mice given protein cages into the lungs.

All patents and publications referred to herein are expressly incorporated by reference in their entirety.

Although the disclosure has been described with reference to the specific embodiments and the foregoing non-limiting examples, it should be understood that various changes and modifications can be made without departing from the spirit of the disclosure.

1. A vaccine composition comprising a protein cage and an adjuvant.

2. The vaccine composition of claim 1, comprising at least two types of a protein cage.

3. The vaccine composition of claim 1, wherein the protein cage is a self-assembled protein cage.

4. The vaccine composition of claim 1, wherein the protein cage is a viral protein cage.

5. The vaccine composition of claim 1, wherein the protein cage is selected from the group consisting of heat shock protein cages, CCMV protein cages, Dps protein cages, and ferritin protein cages.

6. The vaccine composition of claim 1, wherein the protein cage contains one or more mutations.

7. The vaccine composition of claim 1, wherein the protein cage contains a mutation selected from the group consisting of insertions, deletions, substitutions, or a combination thereof.

8. The vaccine composition of claim 1, wherein the protein cage is a heat shock protein cage containing one or more mutations.

9. The vaccine composition of claim 1, wherein the protein cage is a heat shock protein cage with a G41C mutation.

10. The vaccine composition of claim 1, wherein the adjuvant is selected from the group consisting of aluminum salts, endotoxins, and influenza virus-derived membrane-bound hemagglutinin and neuraminidase.

11. A method for inducing an immune protection response against a viral or bacterial infection comprising administering to a subject in need of such treatment an effective amount of a composition comprising a protein cage, wherein the subject develops an immune protection response against the viral or bacterial infection.

12-17. (canceled)

18. The method of claim 11, wherein the protein cage is a heat shock protein cage containing one or more mutations.

19. The method of claim 11, wherein the protein cage is a heat shock protein cage with a G41C mutation.

20-21. (canceled)

22. The method of claim 11, wherein inducing the immune protection response includes developing a localized lymphoid tissue in the subject.

23-31. (canceled)

32. A method of preventing or ameliorating a viral or a bacterial infection comprising administering to a subject in need of such treatment an effective amount of a composition comprising a protein cage, wherein the composition is administered in advance of the viral or the bacterial infection.

33-35. (canceled)

36. The method of claim 32, wherein the viral or bacterial infection is, or is associated with, an infection of the lung.

37. The method of claim 32, wherein the viral infection is an influenza viral infection.

38-42. (canceled)

43. A method of ameliorating asthma in a subject in need thereof, the method comprising administering to the subject an effective amount of a composition comprising a protein cage.

44. The method of claim 43, wherein the protein cage is an HSP protein cage.

45. The method of claim 43, wherein the protein cage comprises one or more mutations.

46-50. (canceled)
